(19) United States
(12) Patent Application Publication
(10) Pub. No.: US 2009/0182208 A1
(43) Pub. Date: Jul. 16, 2009

(54) PHYSIOLOGICAL SIGNAL MEASURING SENSOR AND MANUFACTURING METHOD FOR THE SAME

(76) Inventors: Jae Geol Cho, Yongin-si (KR); Jung Taek Oh, Yongin-si (KR)

Correspondence Address: CHA & REITER, LLC, 210 ROUTE 4 EAST STE 103, PARAMUS, NJ, 07652, US

(21) Appl. No.: 12/350,484

(22) Filed: Jan. 8, 2009

(30) Foreign Application Priority Data
Jan. 10, 2008 (KR) 2008-0002932

(51) Int. Cl. A61B 5/00; H01S 4/00

(52) U.S. Cl. 600/310; 29/592.1

(57) ABSTRACT

A physiological signal measuring sensor that minimizes the measurement error due to a soldering tolerance during the manufacturing process, and a manufacturing method for the same is disclosed. The physiological signal measuring sensor includes a printed circuit board, a light receiving chip mounted to the upper surface of the printed circuit board, light emitting chips mounted to the upper surface of the printed circuit board adjacent to the light receiving chip and resin sealing portions sealing the light receiving chip and the light emitting chips mounted to the upper surface of the printed circuit board wherein a first resin is selected to have opaque optical characteristics and a second resin is selected to have transparent characteristics.

### CLAIM OF PRIORITY

This application claims the benefit of the earlier filing date, pursuant to 35 USC 119, to that patent application entitled “PHYSIOLOGICAL SIGNAL MEASURING SENSOR AND MANUFACTURING METHOD FOR THE SAME” filed in the Korean Intellectual Property Office on Jan. 10, 2008 and assigned Serial No. 2008-0002932, the contents of which is incorporated herein by reference.

### BACKGROUND OF THE INVENTION

The present invention relates to a physiological signal measuring sensor and, more particularly, to a physiological signal measuring sensor that can measure the subcutaneous fat thickness, skin color, and pulse wave of a human body together using light, and a manufacturing method for the same.

Technologies for measuring physiological signals such as pulse waves, skin colors, and subcutaneous fat thicknesses using light are being developed. For example, in order to measure a pulse wave, a near infrared ray is used to measure a change in the amount of light absorbed by hemoglobin caused by a change in the amount of blood and thereby measure a change in flows due to heart beats. In order to measure a skin color, a melanin index of a skin is measured by measuring light absorption rate using a light source having a wavelength near the red color of a high melanin absorption rate and a light source having a wavelength in an infrared ray region insensitive to a change in melanin. In order to measure subcutaneous fat, a body fat percentage is measured by measuring light reflected from a skin to which a near infrared ray is irradiated.

In this manner, a physiological signal measuring sensor is used to measure a physiological signal, and the physiological signal measuring sensor has a structure in which a light emitting device and a light receiving device are mounted to a printed circuit board. Then, the light emitting device and the light receiving device are packaged are mounted onto the printed circuit board by soldering.

In the conventional method of measuring a physiological signal using a physiological signal measuring sensor, a physiological signal is measured by measuring the intensity of light irradiated from a light emitting device and received by a light receiving device attached to a portion of a human body where the physiological signal is measured.

However, since the conventional physiological signal measuring sensor is manufactured by mounting a light emitting device and a light receiving device that are separately manufactured to a printed circuit board, the manufacturing process is complex and there is a limit in maintaining the reliability of such a product. In other words, the light emitting device and the light receiving device soldered to the printed circuit board have undesired height deviations and angle deviations, and as such a measurement error is generated in the conventional physiological signal measuring sensor. In particular, since a skin color measuring sensor is installed close to a light emitting device and a light receiving device, a minute angle change of the light emitting device causes a light path change in a skin layer, generating a severe measurement error.

Moreover, since the conventional physiological signal measuring sensor can measure only one type of physiological signal, more than one physiological signal measuring sensor is necessary to measure different types of physiological signals. This is bothersome to a user.

### SUMMARY OF THE INVENTION

The present invention has been made in view of the above problems, and the present invention provides a physiological signal measuring sensor that has only one device and a manufacturing method for the same.

The present invention also provides a physiological signal measuring sensor that minimizes the measurement error caused by a soldering tolerance during a manufacturing process, and a manufacturing method for the same.

The present invention also provides a physiological signal measuring sensor that maintains measurement accuracy while measuring a plurality of physiological signals and having a reduced size, and a manufacturing method for the same.

In accordance with an exemplary embodiment of the present invention, there is provided a physiological signal measuring sensor comprising a printed circuit board; a light receiving chip mounted to the upper surface of the printed circuit board, light emitting chips mounted to the upper surface of the printed circuit board adjacent to the light receiving chip and resin sealing portions sealing the light receiving chip and the light emitting chips mounted to the upper surface of the printed circuit board, the resin sealing portions including a first resin sealing portion formed by sealing a region including the regions between the light receiving chip and the light emitting chips but excluding the regions in which the light receiving chip and the light emitting chips are mounted with the first sealing resin having a non-transmitting property at a lightwave band of the light emitting chips; and a second resin sealing portion formed in regions where the light receiving chip and the light emitting chips are mounted and sealed with the second sealing resin having a transmitting property at bands of light wavelengths of the light emitting chips.

In accordance with another exemplary embodiment of the present invention, there is provided a manufacturing method for a physiological signal measuring sensor comprising mounting a light receiving chip and light emitting chips to the upper surface of a printed circuit board and sealing a region including the regions between the light receiving chip and the light emitting chips but excluding the regions in which the light receiving chip and the light emitting chips are mounted with a first sealing resin having a non-transmitting property at a light wave band of the light emitting chips, and sealing regions where the light receiving chip and the light emitting chips are mounted with a second sealing resin having a transmitting property at bands of light wavelengths of the light emitting chips.

### BRIEF DESCRIPTION OF THE DRAWINGS

The above and other aspects, features and advantages of certain exemplary embodiments of the present invention will be more apparent from the following detailed description taken in conjunction with the accompanying drawings, in which:

FIG. 1 is a plan view illustrating a physiological signal measuring sensor according to a first embodiment of the present invention;

FIG. 2 is a sectional view taken along line II-II of FIG. 1;

FIG. 3 is a plan view illustrating a physiological signal measuring sensor according to a second embodiment of the present invention;

FIG. 4 is a plan view illustrating a physiological signal measuring sensor according to a third embodiment of the present invention;

FIG. 5 is a plan view illustrating a physiological signal measuring sensor according to a fourth embodiment of the present invention;

FIG. 6 is a plan view illustrating a physiological signal measuring sensor according to a fifth embodiment of the present invention;

FIGS. 7A to 7E are graphs illustrating skin color measurement accuracies of the physiological signal measuring sensors according to the first to fifth embodiments of the present invention;

FIG. 8 is a graph illustrating repetition measurement deviations of skin colors using the physiological signal measuring sensors according to the first to fifth embodiments of the present invention;

FIG. 9 is a plan view illustrating a physiological signal measuring sensor according to a sixth embodiment of the present invention;

FIGS. 10 to 13 are views illustrating a manufacturing method for the physiological signal measuring sensors according to the first to sixth embodiments of the present invention;

FIGS. 10 and 11 are a plan view and a sectional view illustrating a mounting step;

FIG. 12 is a sectional view illustrating a first resin sealing portion forming step;

FIG. 13 is a sectional view illustrating a second resin sealing portion forming step;

FIGS. 14 and 15 are views illustrating a manufacturing method for a physiological signal measuring sensor according to a seventh embodiment of the present invention; and

FIGS. 16 and 17 are views illustrating a manufacturing method for a physiological signal measuring sensor according to an eighth embodiment of the present invention.

### DETAILED DESCRIPTION OF THE INVENTION

Exemplary embodiments of the present invention are described in detail with reference to the accompanying drawings. It should be understood that the same elements are represented by the same reference numerals in the accompanying drawings. In addition, only parts necessary to understand operation of the embodiment of the present invention will be described, and others will be omitted to avoid obscuring the subject matter of the present invention.

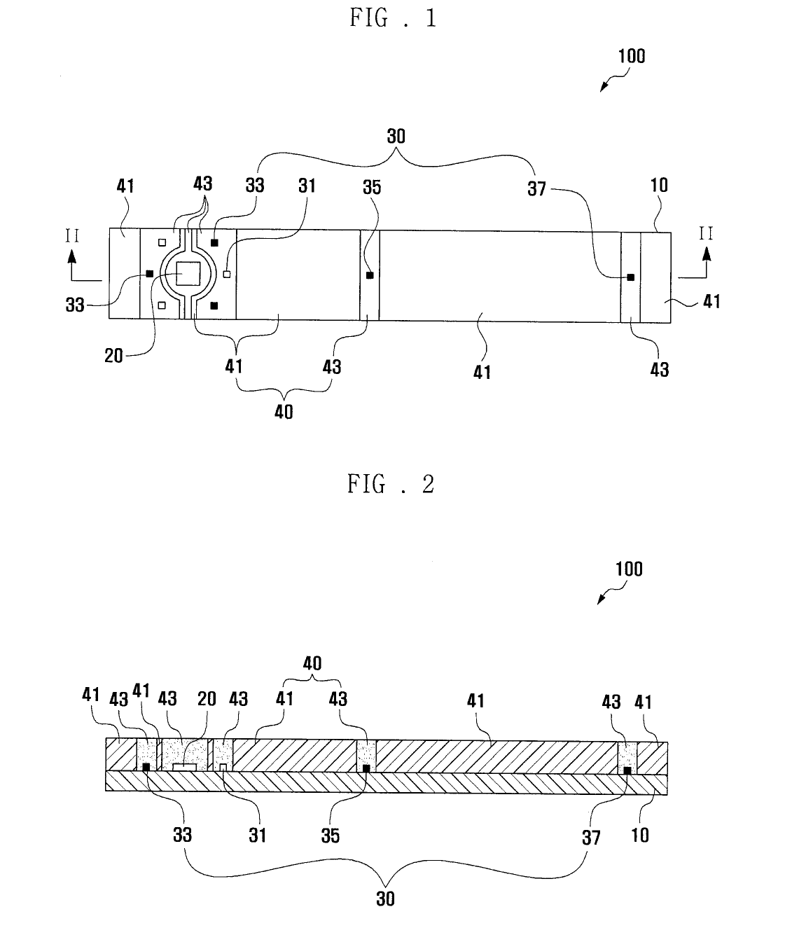

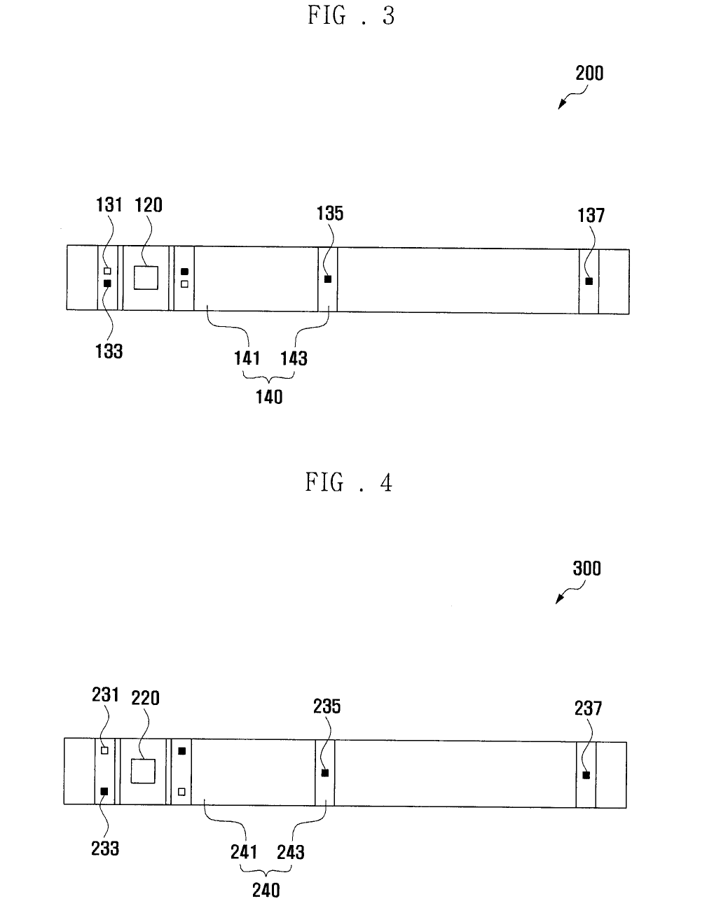

As illustrated in FIGS. 1 and 2, a physiological signal measuring sensor 100 according to the first embodiment of the present invention has a structure in which a light receiving chip 20 and light emitting chips 30 are mounted onto the upper surface of a printed circuit board 10 and are sealed by resin sealing portions 40 formed of sealing resins. In other words, the physiological signal measuring sensor 100 according to the first embodiment of the present invention has a multi-chip package configuration in which the light receiving chip 20 and the light emitting chips 30 are mounted together inside the resin sealing portions 40.

The light receiving chip 20 is attached to one side of the upper surface of the printed circuit board 10. The light emitting chips 30 are attached to the upper surface of the printed circuit board 10 adjacent to the light receiving chip 20. Some portions of the upper surfaces of the light receiving chip 20 and the light emitting chips 30 are electrically connected to the printed circuit board 10 through bonding wires (not shown).

The resin sealing portion 40 includes first resin sealing portions 41 and second resin sealing portions 43. The first resin sealing portions 41 are formed by sealing a region including the regions between the light receiving chip 20 and the light receiving chips 30, but excluding the regions in which the light receiving chip 20 and the light emitting chips 30 are mounted, with a first sealing resin having a non-transmitting property at a lightwave band of the light emitting chips 30.

Since the regions in which the light receiving chip 20 and the light emitting chips 30 are sealed with the second resin sealing portions 43 and the remaining regions are sealed with the first resin sealing portions 41, almost all the light emitted from the light emitting chip 30 is irradiated to a portion of a human body that is to be measured through the second resin sealing portions 43. Hence, the light emitted from the light emitting chips 30 that does not pass through the portion of the human body and directly faces the light receiving chip 20 is interrupted, thereby enhancing the signal to noise ratio (SNR) and increasing measurement accuracy.

Since the light receiving chip 20 and the light emitting chips 30 are directly mounted to the printed circuit board 10 and are sealed by the resin sealing portions 40, the heights and angles of the resin sealing portions 40 contacted with a skin are all substantially identical. Hence, the physiological signal measuring sensor 100 according to the first embodiment of the present invention minimizes the measurement deviation of the product.

The physiological signal measuring sensor 100 according to the first embodiment of the present invention is now described in detail.

The printed circuit board 10 is a rectangular wire board, and its transverse length is longer than its longitudinal length.

The light receiving chip 20 is mounted to one side of the upper surface of the printed circuit board 10, and receives the light reflected by the portion of the human body and measures a physiological signal by detecting the intensity of the received light. A photodiode is generally used as the light receiving chip 20. Although only a single light receiving device is shown, it would be recognized that the light receiving device may consist of at least one light receiving device.

The light emitting chips 30 irradiate light to a desired portion of a human body. Light emitting diodes (LED) that emit light of wavelengths in the visible ray region or the infrared ray region are generally used as the light emitting chips 30. In particular, the light emitting chip 30 includes first and second light emitting chips 31 and 33 mounted around the light receiving chip 20, and at least one third light emitting chip 35 mounted further away from the light receiving chip 20 than the first and second light emitting chips 31 and 33, and at least one fourth light emitting chip 37 mounted further away form the light receiving chip 20 than the third light emitting chip 35.

The first and second light emitting chips 31 and 33 are symmetrically disposed on opposite sides of the light receiving chip 20 and are spaced apart from the light receiving chip 20 by the same distance. The distance between the light receiving chip 20 and the first and second light emitting chips 31 and 33 is, in a preferred embodiment is in the order of 1 to 5 millimeter (mm). In the first embodiment of the present invention, three first light emitting chips 31 and three second light emitting chips 33 are symmetrically disposed (in distance) on opposite sides of the light receiving chip 20 and are spaced apart from the light receiving chip 20 by the same distance. The first light emitting chips 31 emit light of wavelengths of in a visible light region, and the second light emitting chips 33 emit light of wavelengths in a near infrared light region.

The third light emitting chip 35 is used to measure pulse waves, and is spaced apart form the light receiving chip 20 by a distance in the order 3 to 15 mm. The third light emitting chip 35 emits light of a wavelength of a near infrared ray region.

The fourth light emitting chip 37 is used to measure subcutaneous fat thickness, and is spaced apart from the light receiving chip 20 by a distance in the order of 10 to 50 mm. The subcutaneous fat thickness is measured using the skin color data obtained by the first and second light emitting chips 31 and 33. In other words, since the collagen fiber contained in a skin of a human body has asymmetrical optical characteristics, in order to measure the subcutaneous fat thickness and skin color exactly, a number of the first and second light emitting chips 31 and 33 are arranged around the light receiving chip 20 circularly and alternately so that light irradiated by the emitting chips 31 and 33 can be received by the light receiving chip 20 regardless of contact direction of the physiological signal measuring sensor 100 to the skin.

Hence, in the first embodiment of the present invention, the first and second light emitting chips 31 and 33 are symmetrically disposed around the light receiving chip 20 and are spaced apart from the light receiving chip 20 by the same distance.

Meanwhile, since the fourth light emitting chip 37 is installed on one side of the light receiving chip 20, it is necessary to maintain the directionality of the physiological signal measuring sensor 100 in order to perform a uniform measurement. Since the intensity of light measured by the light receiving chip 20 decreases as a function of the distance between the fourth light emitting chip 37 and the light receiving chip 20 increases, a plurality of fourth light emitting chip 37 may be spaced apart from the light receiving chip 20 by the same distance to obtain a sufficient intensity of light.

The resin sealing portions 40 may be formed by transfer molding. The first and second sealing resins may be formed of an epoxy resin. The first sealing resin may be an opaque epoxy resin and the second sealing resin may be a transparent epoxy resin.

In particular, the first and second resin sealing portions 41 and 43 have the same height. This is because measuring accuracy is increased by preventing contact areas of the first and second resin sealing portions from being varied according to measurement pressures when the first and second resin sealing portions 41 and 43 make contact with measured portions of a human body.

The first resin sealing portions 41 are formed between the first and second light emitting chips 31 and 33 and the light receiving chip 20. The first resin sealing portions 41 are also formed between the first and second light emitting chips 31 and 33 and the third light emitting chip 35. The first resin sealing portions 41 are also formed between the third light emitting chip 35 and the fourth light emitting chip 37.

The second resin sealing portions 43 are formed in regions where the light receiving chip 20 is mounted. The second resin sealing portions 43 are also formed at regions where the first and second light emitting chips 31 and 33 are mounted on a side of the light receiving chip 20 and at regions where the first and second light emitting chips 31 and 33 are mounted on the opposite sides of the light receiving chip 20. The second resin sealing portions 43 are formed because the regions where the first and second light emitting chips 31 and 33 are mounted are divided by the first resin sealing portions 43 formed on opposite sides of the light receiving chip 20. The second resin sealing portions 43 are also formed in a region where the third light emitting chip 35 is mounted and in a region where the fourth light emitting chip 37 is mounted.

Hence, after the light emitted from the first to fourth light emitting chips 31, 33, 35, and 37 is projected onto the skin contacted with the resin sealing portions 40, it is introduced into the light receiving chip 20 from the skin by multiple scattering.

Meanwhile, the first and second light emitting chips 31 and 33 are disposed around the light receiving chip 20, and the third and fourth light emitting chips 35 and 37 are disposed on the same line as the light receiving chip 20 and are spaced apart from the light receiving chip 20 by predetermined distances. Hence, since the second resin sealing portions 43, sealing the regions where the light receiving chip 20 and the light emitting chips 30 are formed, are discontinuously formed and the first resin sealing portions 41 are formed between the second resin sealing portions 43, the first and second resin sealing portions 41 and 43 may have band-shapes.

Although the first and second light emitting chips 31 and 33 are spaced apart from the light receiving chip 20 by the same distance in the first embodiment of the present invention, the present invention is not limited thereto. For example, first and second light emitting chips may be symmetrically disposed on opposite sides of a light receiving chip as illustrated in FIGS. 3 to 6.

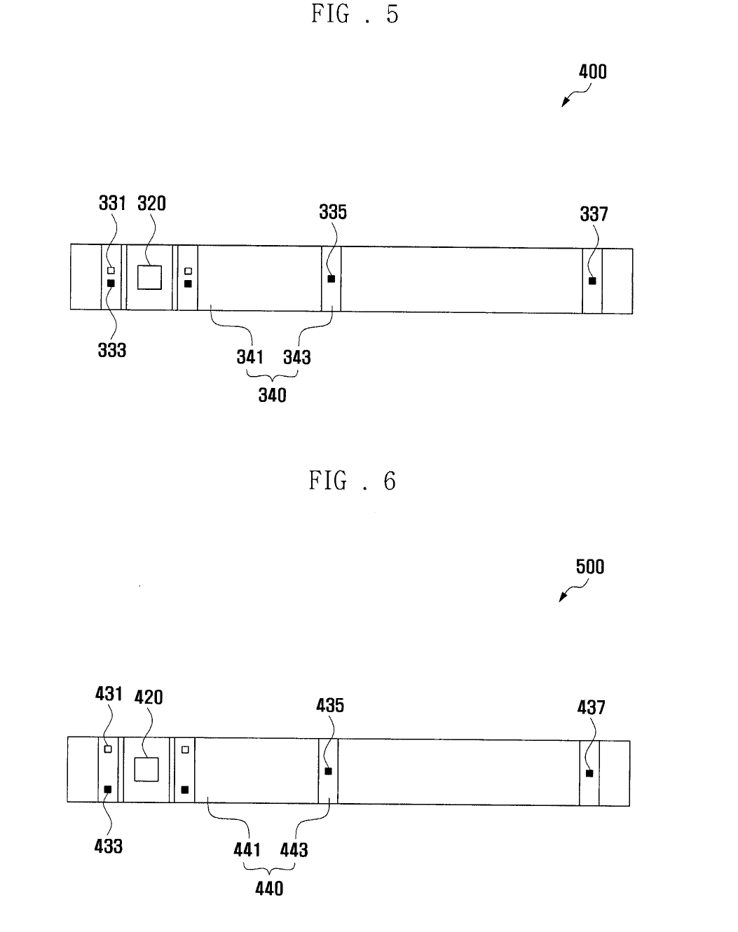

As illustrated in FIGS. 3 and 4, the physiological signal measuring sensors 200 and 300 according to the second and third embodiments of the present invention have structures in which two first light emitting chips 131 and 231 (FIGS. 3 and 4, respectively) and two second light emitting chips 133 and 233 (FIGS. 3 and 4, respectively) are symmetrically disposed on opposite sides of light receiving chips 120 and 220. Third light emitting chips 135 and 235 (FIGS. 3 and 4, respectively) and fourth light emitting chips 137 and 237 (FIGS. 3 and 4, respectively) are disposed as in the physiological signal measuring sensor 100 according to the first embodiment of the present invention, so a detailed description thereof will be omitted.

The first light emitting chips 131 and 231 and the second light emitting chips 133 and 233 are disposed on opposite sides of the light receiving chips 120 and 220 respectively, and the lines connecting the two first light emitting chips 131 and 231 cross the lines connecting the two second light emitting chips 133 and 233 at the light receiving chips 120 and 220.

Then, the first light emitting chips 131 and 231 and the second light emitting chips 133 and 233 are close to each other in the second embodiment of the present invention, and are spaced apart from each other in the third embodiment of the present invention.

As illustrated in FIGS. 5 and 6, the physiological signal measuring sensors 400 and 500 according to the fourth and fifth embodiments of the present invention have structures in which two first light emitting chips 331 and 431 (FIGS. 5 and 6, respectively) and two second light emitting chips 333 and 433 (FIGS. 5 and 6, respectively) are symmetrically disposed on opposite sides of light receiving chips 320 and 420. The third light emitting chips 335 and 435 and the fourth light emitting chips 337 and 437 are disposed as in the physiological signal sensor 100 according to the first embodiment of the present invention, and a detailed description thereof will be omitted.

The first light emitting chips 331 and 431 and the second light emitting chips 333 and 433 are disposed on opposite sides of the light receiving chips 320 and 420 respectively, and the lines connecting the two first light emitting chips 331 and 431 and the lines connecting the two second light emitting chips 333 and 433 are disposed parallel to each other.

Then, the first light emitting chips 131 and 231 and the second light emitting chips 133 and 233 are close to each other in the fourth embodiment of the present invention, and are spaced apart from each other in the fifth embodiment of the present invention.

In the second to fifth embodiments of the present invention, in order to minimize the sizes of the physiological signal measuring sensors 200, 300, 400, and 500, the numbers of the first and second light emitting chips 131 and 133 mounted around the light receiving chip 120 are minimized as illustrated in FIG. 3. Moreover, the first and second light emitting chips 131 and 133 are symmetrically disposed on opposite sides of the light receiving chip 120 instead of being circumferentially disposed at a constant radius about the light receiving chip 120.

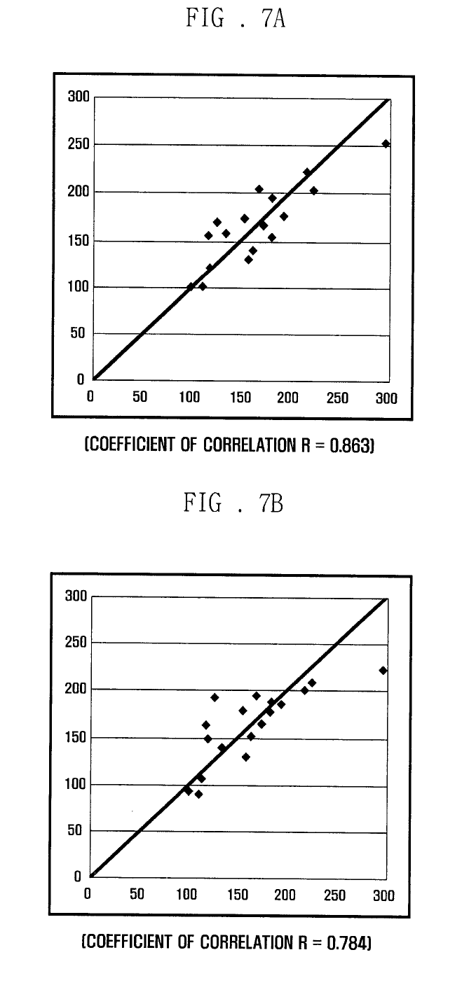

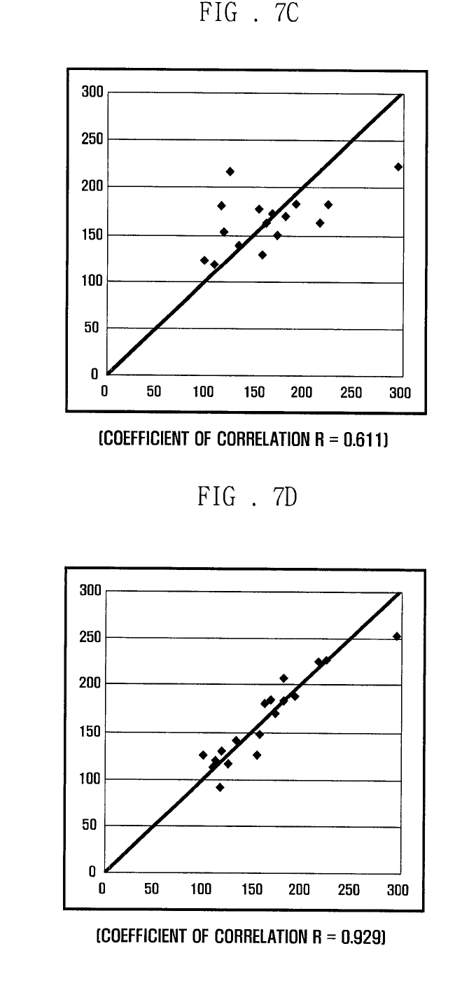

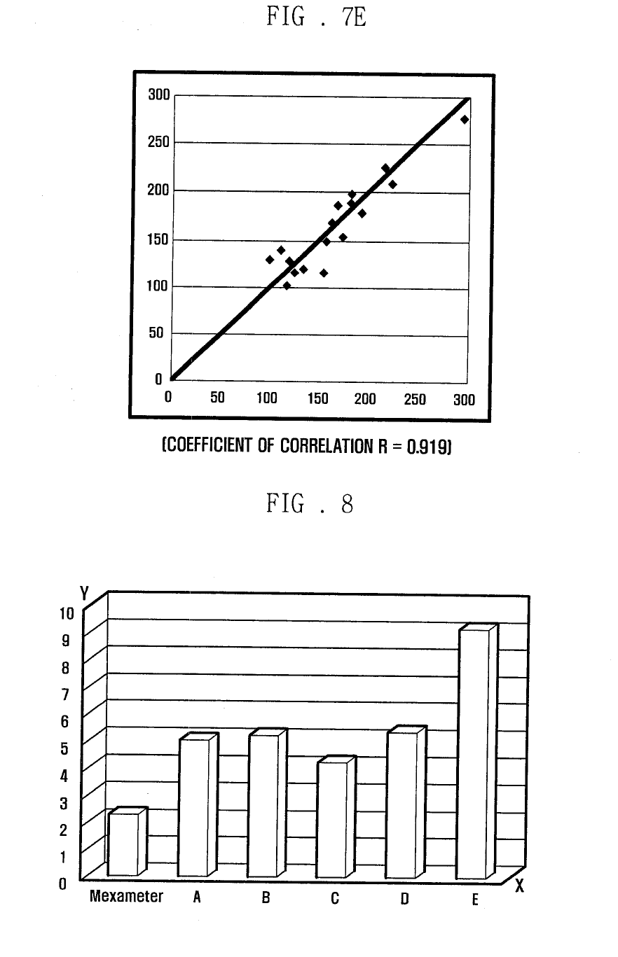

Skin color measurement accuracies and repetitive measurement deviations of the physiological signal measuring sensors 100, 200, 300, 400, and 500 according to the first to fifth embodiments of the present invention will be described with reference to FIGS. 7A to 7E and 8. Then, the standard devices used to compare the skin color measurement accuracies and repetitive measurement deviations are a Mexameter MX18 model of CK electronic.

Referring to FIGS. 7A to 7E, the skin color measurement accuracies R of the physiological signal measuring sensors 100, 200, 300, 400, and 500 according to the first to fifth embodiments of the present invention are 0.863, 0.784, 0.611, 0.929, and 0.919 respectively. In other words, it can be seen that the physiological signal measuring sensors 400 and 500 according to the fourth and fifth embodiments of the present invention have the same level of accuracies as the physiological signal measuring sensor 100 according to the first embodiment of the present invention.

Referring to FIG. 8, the standard deviations of the physiological signal measuring sensors 100, 200, 300, 400, and 500 according to the first to fifth embodiments of the present invention that are obtained by three repetitive measurements, respectively, are represented in the order of letters A to E and it can be seen that the repetitive performances become better as the values of Y axis in FIG. 8 become lower.

Referring to FIG. 5, for example, in order to minimize the sizes of the physiological signal measuring sensors 200, 300, 400, and 500 according to the second to fifth embodiments of the present invention, when the first and second light emitting chips 331 and 333 are disposed, given the lines connecting the light emitting chips of the same wavelength are drawn about the light receiving chip 320, the line connecting the two first light emitting chips 331 and the line connecting the two second light emitting chips 333 are preferably disposed parallel to each other. In addition, it is advantageous in repetitive performances to make the first and second light emitting chips 331 and 333 as close as possible to each other.

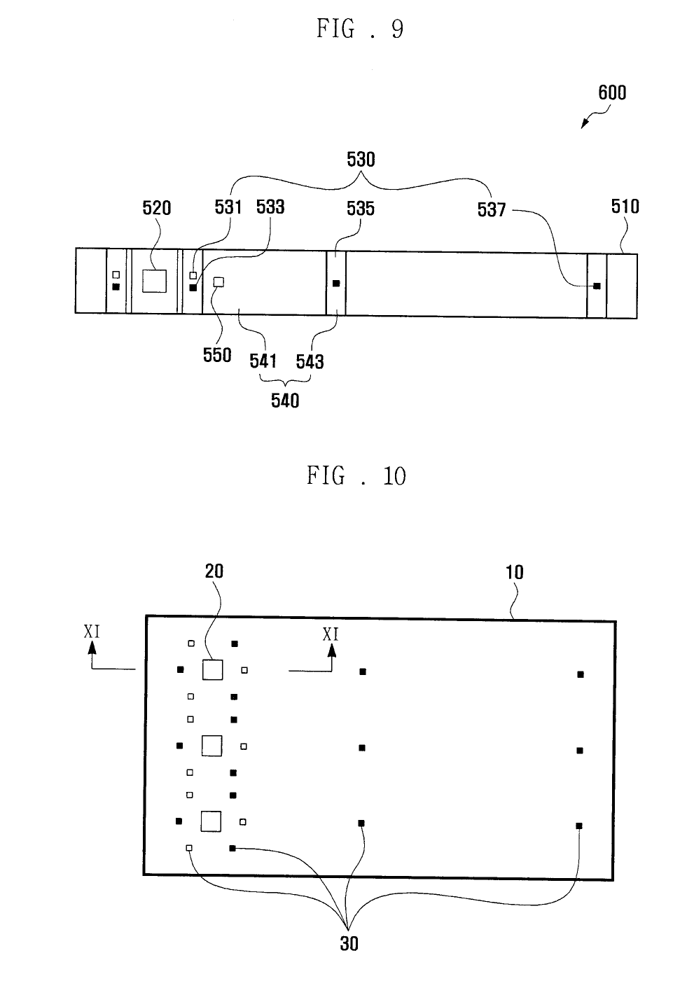

Meanwhile, since outputs of light emitting chips are varied according to the temperature of the environment where a physiological signal measuring sensor is located, as illustrated in FIG. 9, a physiological signal measuring sensor 600 according to the sixth embodiment of the present invention may further include a temperature compensation device 550 that measures the temperature of the environment where it is used.

The temperature compensation device 550 is mounted to the upper surface of a printed circuit board 510 and may be sealed by a first resin sealing portion 541. In particular, the temperature compensation device 550 is preferably disposed close to the first and second light emitting chips 531 and 533 that measure a skin color whose value is severely changed according to the temperature of the physiological signal measuring sensor 600. In other words, the temperature compensation device 550 is mounted to the printed circuit board 510 between the first and second light emitting chips 531 and 533 and the third light emitting chip 535, and is preferably mounted onto the upper surface of the printed circuit board 510 adjacent to the first and second light emitting chips 531 and 533.

When measuring a physiological signal, the physiological signal measuring sensor 600 according to the sixth embodiment of the present invention compensates for an output of the light emitting chip 530 or a physiological signal value using a temperature measured by the temperature compensation sensor 550.

The manufacturing methods for the physiological signal measuring sensors according to the first to sixth embodiments of the present invention will be described in detail with reference to FIGS. 10 to 13. As the physiological signal measuring sensors according to the first to sixth embodiments of the present invention are manufactured by the same manufacturing method, the manufacturing method for the physiological signal measuring sensor according to the first embodiment of the present invention will be mainly described.

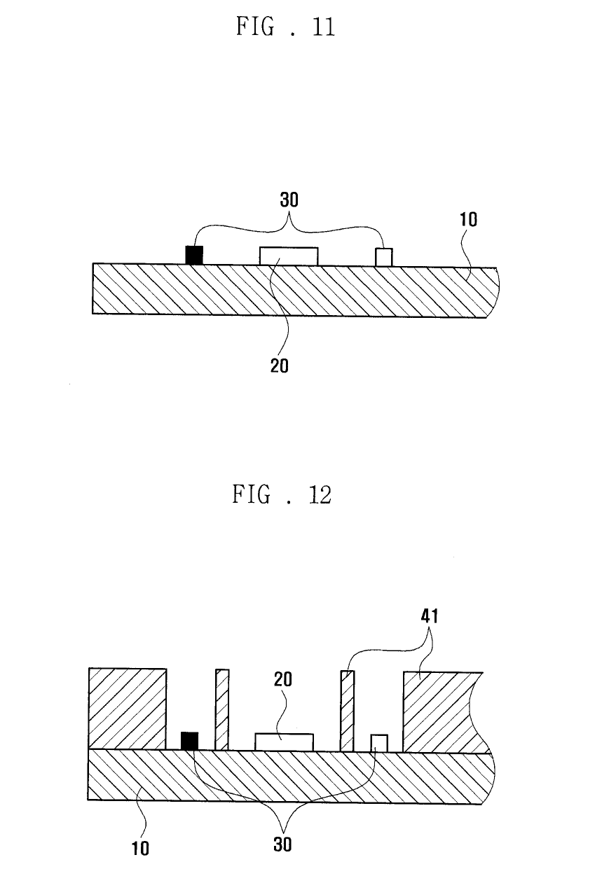

First, as illustrated in FIGS. 10 and 11, the light receiving chips 20 and the light emitting chips 30 are mounted onto the upper surface of the printed circuit board 10. Then, the printed circuit board 10 has a strip configuration that enables collective manufacturing of a plurality of physiological signal measuring sensors.

As illustrated in FIGS. 12 and 13, resin sealing portions 40 sealing the light receiving chips 20 and the light emitting chips 30 mounted onto the upper surface of the printed circuit board 10 are formed. The light receiving chips 20 and the light emitting chips 30 may be sealed by a transfer molding method.

In particular, after the first sealing process of forming the first resin sealing portions 41, a second sealing process for forming the second resin sealing portions 43 is performed.

As illustrated in FIG. 12, in the first sealing process, the first resin sealing portions 41 are formed by sealing a region including the regions between the light receiving chip 20 and the light receiving chips 30, but excluding the regions in which the light receiving chip 20 and the light emitting chips 30 are mounted with the first sealing resin. As illustrated in FIG. 13, the second resin sealing portions 43 are formed by sealing a region where the light receiving chips 20 and the light emitting chips 30 are mounted with the second sealing resin. In other words, the second sealing process is a process of forming the second resin sealing portions 43 by filling regions between the first resin sealing portions 41 with the second sealing resin after forming the first resin sealing portions 41. Then, in the sealing process of the first manufacturing method, the heights of the first resin sealing portions 41 and the second resin sealing portions 43 are the same.

Finally, the stripped printed circuit board 10 having the resin sealing portions 40 is separated into the individual physiological signal measuring sensors 100.

Although the second resin sealing portions 43 are formed after the formation of the first resin sealing portions 41 in the manufacturing method, the first resin sealing portions 41 may be formed after the formation of the second resin sealing portions 43.

In addition, although the first resin sealing portions 41 and the second resin sealing portions 43 are formed to be band-shaped in the manufacturing method, the present invention is not limited thereto. For example, the second resin sealing portions 43 sealing the light receiving chips 20 and the light emitting chips 30 are formed in the form of islands, and the first resin sealing portions 41 may be formed in the remaining regions.

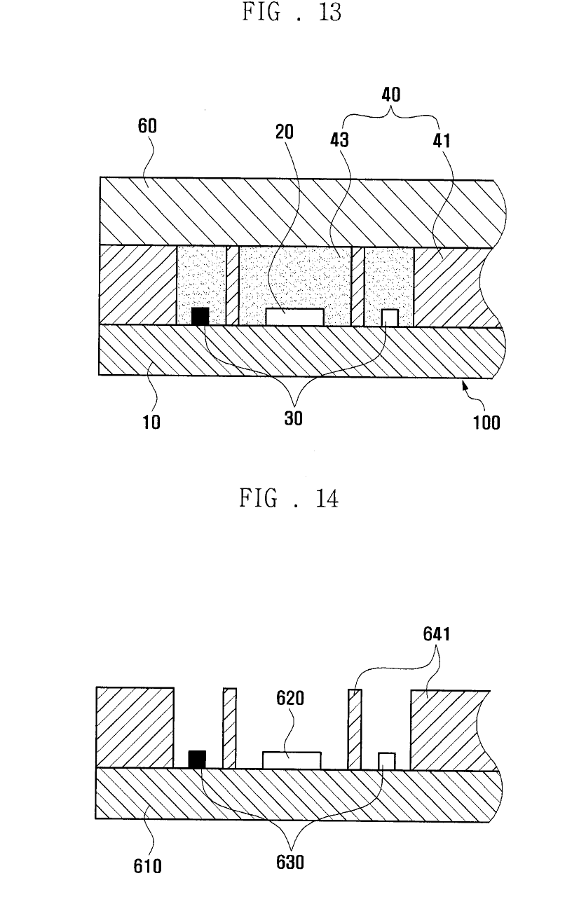

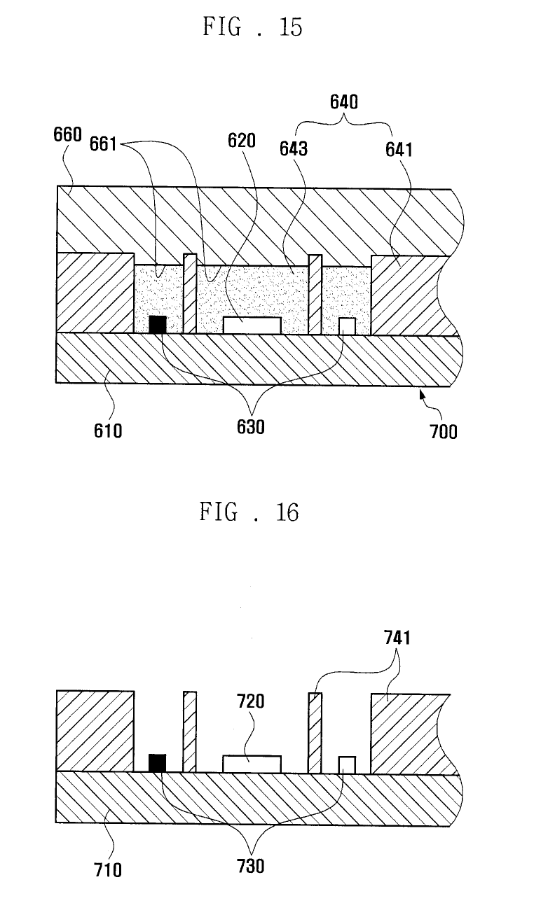

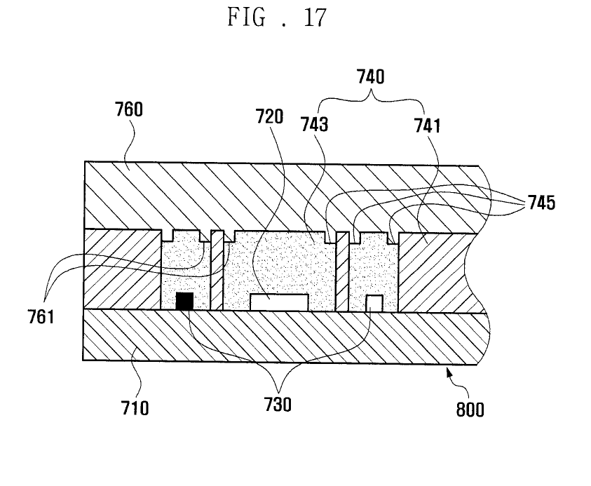

Although the first resin sealing portions and the second resin sealing portions have the same height in the first to sixth embodiments of the present invention, in the seventh and eighth embodiments of the invention, the first resin sealing portions and the second resin sealing portions have different heights. For example, FIGS. 14 and 15 represent a seventh embodiment of the invention and FIG. 17 showing grooves 745 that may be formed along the borders of the first and second resin sealing portions 741 and 743 represents an eighth embodiment of the invention claimed.

Referring to FIGS. 14 and 15, in the physiological signal measuring sensor 700 according to the seventh embodiment of the present invention, the first resin sealing portions 641 are higher than the second resin sealing portions 643. The first and second resin sealing portions 641 and 643 have different heights to prevent the second liquefied sealing resin from penetrating to the upper surface of the first resin sealing portions when the second resin sealing portions 643 are formed after the formation of the first resin sealing portions 641. Since the refraction index of a transparent epoxy resin is generally higher than that of the air, some light emitted inside the second resin sealing portions 643 is generally reflected, in total, at the border surfaces of the second resin sealing portions 643 and the air. Hence, when the liquefied second sealing resin intrudes to the upper surfaces of the first resin sealing portions 641, the light emitted from the light emitting chips 630 is wave-guided by the transparent second sealing resin, thus, changing the means of introduction of light into a skin and causing a measurement error.

Then, the steps of the first and second resin sealing portions 641 and 643 are not large so that a measured skin portion can make stable contact with the second resin sealing portions 643.

In order to make the heights of the first and second resin sealing portions 641 and 643 different, in a mold 660 used in the second sealing process, bosses 661 are formed lower than the height of the first resin sealing portions 641.

Although the first resin sealing portions 641 are higher than the second resin sealing portions 643 in the seventh embodiment of the present invention, the second resin sealing portions may be higher. In other words, when the first resin sealing portions are formed after the formation of the second resin sealing portions, the first sealing resin is restrained from intruding to the upper surfaces of the second resin sealing portions.

Referring to FIGS. 16 and 17, grooves 745 are formed along the borders of the first and second resin sealing portions 741 and 743 in the physiological signal measuring sensor 800 according to the eighth embodiment of the present invention.

In other words, the grooves 745 may be formed along the borders of the first and second resin sealing portions 741 and 743 by forming bosses 761 at the borders of the first and second resin sealing portions 741 and 743 using a mold 760 used in the second sealing process.

Meanwhile, in the seventh embodiment of the present invention, since the heights of the first and second resin sealing portions are different, the areas where the resin sealing portions having a low height make contact with a measured portion of a human body are varied according to measurement pressure. However, since the heights of the first and second resin sealing portions 741 and 743 are the same in the eighth embodiment of the present invention, an influence due to the measurement pressure can be minimized and an error due to repetitive measurements can be also minimized.

In the physiological signal measuring sensor 800 according to the eighth embodiment of the present invention, the first and second resin sealing portions 741 and 743 have the same height so as to restrain the liquefied second sealing resin from penetrating in the upper surface of the first resin sealing portions 741.

Although the grooves 745 are formed in the second resin sealing portions 743 proximate to the first resin sealing portions 741 in the eighth embodiment of the present invention, they may be formed in the first resin sealing portions 741 proximate to the second resin sealing portions 743 when the second resin sealing portions 743 are formed first.

Since the physiological signal measuring sensor according to the present invention has a multi-chip package configuration in which light emitting chips and light receiving chips are mounted to a printed circuit board and then are sealed by an epoxy resin, it takes the form of one device. In particular, since regions where light emitting chips and light receiving chips are sealed by transparent epoxy resins and the remaining regions are sealed by an opaque epoxy resin, the light emitting from the light emitting chips to first resin sealing portions formed of an opaque epoxy resin are interrupted, and almost all the light is irradiated to a measured portion of a human body through second resin sealing portions formed of an opaque epoxy resin. Hence, the light emitted from the light emitting chips 30 that does not pass through the portion of the human body and directly faces the light receiving chips is interrupted, thereby enhancing signal to noise ratio (SNR) and increasing measurement accuracy.

Since the physiological signal measuring sensor according to the present invention takes the form of one package, height deviations and angle deviations of light emitting chips and light receiving chips can be minimized, thereby minimizing a measurement error (deviation) of a product.

Since the physiological signal measuring sensor according to the present invention has a structure in which light emitting chips necessary for generating each of a plurality of physiological signals are disposed about light receiving chips, a plurality of physiological signals can be measured together. In particular, since only two first light emitting chips and two second light emitting chips are mounted around light receiving chips and the first and second light emitting chips are symmetrically disposed on opposite sides of the light receiving chips, measurement accuracy can be maintained and the size of the physiological signal measuring sensor can be minimized. Accordingly, the physiological signal measuring sensor according to the present invention is easily portable, and can be applied to a plurality of available, low cost devices, e.g., a mobile phone, a personal digital assistant (PDA), a portable multimedia player (PMP), and an MPEG layer 3 (MP3) player.

In addition, a liquefied sealing resin of resin sealing portions formed later is restrained from penetrating the upper surfaces of first formed resin sealing portions, by forming grooves at the borders of the first resin sealing portions and the second resin sealing portions and making the heights of the first and second resin sealing portions same. Accordingly, an error due to repetitive measurements and measurement pressures can be minimized while maintaining the current level of difficulty in manufacturing the physiological signal measuring sensor.

Although exemplary embodiments of the present invention have been described in detail hereinabove, it should be understood that many variations and modifications of the basic inventive concept herein described, which may appear to those skilled in the art, will still fall within the spirit and scope of the exemplary embodiments of the present invention as defined in the appended claims.

What is claimed is:

1. A physiological signal measuring sensor comprising:
a printed circuit board;
a light receiving chip mounted to the upper surface of the printed circuit board;
light emitting chips mounted to the upper surface of the printed circuit board adjacent to the light receiving chip; and
resin sealing portions sealing the light receiving chip and the light emitting chips mounted to the upper surface of the printed circuit board,
the resin sealing portions including:
first resin sealing portions formed by sealing a region including the regions between the light receiving chip and the light emitting chips, but excluding the regions in which the light receiving chip and the light emitting chips are mounted, with a first sealing resin having a non-transmitting property at a wavelength band of the light emitting chips; and
second resin sealing portions formed in regions where the light receiving chip and the light emitting chips are mounted and sealed with a second sealing resin having a transmitting property at bands of light wavelengths of the light emitting chips.

2. The physical signal measuring sensor according to claim 1, wherein the heights of the first and second resin sealing portions are the same.

3. The physical signal measuring sensor according to claim 2, wherein grooves are formed along the borders of the first and second resin sealing portions.

4. The physiological signal measuring sensor according to claim 3, wherein the light emitting chips includes:
first and second light emitting chips mounted around the light receiving chip;
at least one third light emitting chip spaced apart from the light receiving chip further than the first and second light emitting chips; and
at least one fourth light emitting chip spaced apart from the light receiving chip further than the third light emitting chip.

5. The physiological signal measuring sensor according to claim 4, wherein the first resin sealing portions are formed:
between the first and second light emitting chips and the light receiving chip,
between the first and second light emitting chips and the third light emitting chip, and
between the third light emitting chip and the fourth light emitting chip.

6. The physiological signal measuring sensor according to claim 5, wherein the first and second light emitting chips are symmetrically disposed on opposite sides of the light receiving chip.

7. The physiological signal measuring sensor according to claim 6, wherein the numbers of the first and second light emitting chips are two respectively, and a line connecting the two first light emitting chips and a line connecting the two second light emitting chips are disposed in parallel to each other.

8. The physiological signal measuring sensor according to claim 7, wherein the first and second light emitting chips disposed on opposite sides of the light receiving chip are adjacent to each other.

9. The physiological signal measuring sensor according to claim 4, wherein the first light emitting chips emit light of wavelengths in a visible region, and the second, third, and fourth light emitting chips emit light of wavelengths in a near-infrared region.

10. The physiological signal measuring sensor according to claim 9, further comprising:
a temperature compensation device mounted to the printed circuit board between the first and second light emitting chips and the third light emitting chip and sealed by the first resin sealing portion.

11. The physiological signal measuring sensor according to claim 1, wherein the heights of the first and second resin sealing portions are different.

12. The physiological signal measuring sensor according to claim 1, wherein the first and second sealing resins are epoxy resins.

13. The physiological signal measuring sensor according to claim 12, wherein the first resin sealing portions are opaque and the second resin sealing portions are transparent.

14. The physiological signal measuring sensor according to claim 12, wherein the resin sealing portions are formed by a transfer molding method.

15. A manufacturing method for a physiological signal measuring sensor comprising:
mounting a light receiving chip and light emitting chips to the upper surface of a printed circuit board; and
sealing a region including the regions between the light receiving chip and the light emitting chips but excluding the regions in which the light receiving chip and the light emitting chips are mounted with a first sealing resin having a non-transmitting property at a light wave band of the light emitting chips, and sealing regions where the light receiving chip and the light emitting chips are mounted with a second sealing resin having a transmitting property at bands of light wavelengths of the light emitting chips.

16. The manufacturing method according to claim 15, wherein the sealing process includes:
sealing first sealing portions with the first sealing resin; and
sealing second sealing portions with the second sealing resin.

17. The manufacturing method according to claim 16, wherein the heights of the first and second resin sealing portions are the same.

18. The manufacturing method according to claim 17, wherein grooves are formed along the borders of the first and second resin sealing portions.

19. The manufacturing method according to claim 18, wherein the light emitting chips includes:
first and second light emitting chips mounted around the light receiving chip;
at least one third light emitting chip spaced apart from the light receiving chip further than the first and second light emitting chips; and
at least one fourth light emitting chip spaced apart from the light receiving chip further than the third light emitting chip.

20. The manufacturing method according to claim 19, wherein the first resin sealing portions are formed between the first and second light emitting chips and the light receiving chip, are formed between the first and second light emitting chips and the third light emitting chip, and are formed between the third light emitting chip and the fourth light emitting chip.

21. The manufacturing method according to claim 20, wherein the first and second light emitting chips are symmetrically disposed on opposite sides of the light receiving chip.

22. The manufacturing method according to claim 21, wherein the numbers of the first and second light emitting chips are two respectively, and a line connecting the two first light emitting chips and a line connecting the two second light emitting chips are disposed in parallel to each other.

23. The manufacturing method according to claim 22, wherein the first and second light emitting chips disposed on opposite sides of the light receiving chip are adjacent to each other.

24. The manufacturing method according to claim 20, wherein the first light emitting chips emit light of wavelengths in a visible light region, and the second, third, and fourth light emitting chips emit light of wavelengths in a near infrared light region.

25. The manufacturing method according to claim 24, further comprising:
mounting a temperature compensation device to the printed circuit board between the first and second light emitting chips and the third light emitting chip.
